(19) United States
(12) Patent Application Publication
(10) Pub. No.: US 2019/0348173 A1
(43) Pub. Date: Nov. 14, 2019

(54) HOSPITAL SUPPORT SYSTEM, HOSPITAL SUPPORT METHOD, CONTROL DEVICE, AND CONTROL PROGRAM

(71) Applicant: FUJIFILM Corporation, Tokyo (JP)

(72) Inventors: Shigetoshi ISHIKAWA, Kanagawa (JP); Hirona YUMBE, Kanagawa (JP); Akemi ODA, Kanagawa (JP); Yasuhisa KANEKO, Kanagawa (JP); Keiji TSUBOTA, Kanagawa (JP)

(73) Assignee: FUJIFILM Corporation, Tokyo (JP)

(21) Appl. No.: 16/404,755

(22) Filed: May 7, 2019

(30) Foreign Application Priority Data
May 11, 2018 (JP) 2018-092394

(51) Int. Cl. G16H 40/20

(52) U.S. Cl. CPC G16H 40/20

(57) ABSTRACT

A hospital support system includes an electronic pen that includes a storage unit which stores obtained diseased animal information, an obtaining unit which obtains a position within an animal hospital in which the diseased animal information is obtained, and a transmission unit which transmits the diseased animal information stored in the storage unit and positional information obtained by the obtaining unit and a controller of an operation terminal that performs control for storing, as hospital management information, the diseased animal information in association with identification information for identifying a subject for each diseased animal in a storage unit of the operation terminal based on the positional information transmitted from the transmission unit of the electronic pen.

### CROSS-REFERENCE TO RELATED APPLICATIONS

This application claims priority from Japanese Patent Application No. 2018-092394, filed May 11, 2018, the disclosure of which is incorporated herein by reference in its entirety.

### BACKGROUND

### 1. Technical Field

The present disclosure relates to a hospital support system, a hospital support method, a control device, and a control program.

### 2. Related Art

In the related art, in a hospital in which medical care is performed on a person or an animal hospital in which medical care is performed on an animal, since there is a concern that a device will be broken or contaminated due to the rampage of a subject such as an infant or an animal, an electronic device such as a tablet terminal or a personal computer is requested not to be used in a check-up room. Thus, a wearable device such as an electronic pen or a camera attachable to eyeglasses has been used as a simple electronic device having a little concern that the device will be broken or contaminated due to the subject.

For example, JP2004-054375A describes a technology in which in a case where a doctor writes necessary items on a medical chart sheet on which patterns such as ruled lines are printed on a mount of sheets on which dot patterns are printed by using an electronic pen, written data written by using the electronic pen and identification information such as a medical chart number in association with each other are stored in a medical chart management device.

However, the wearable device is simple and easily carried, whereas a place in which the doctor uses the wearable device for medical care, such as a check-up room or an examination room is not known.

The present disclosure has been made in view of the aforementioned circumstances, and an object of the present disclosure is to provide a hospital support system, a hospital support method, a control device, and a control program capable of easily recognizing a position in which information on a subject is obtained by a wearable device.

### SUMMARY

In order to achieve the object, a hospital support system of a first aspect of the present disclosure comprises: a wearable device that includes a storage unit which stores obtained subject information on a subject, an obtaining unit which obtains a position within a hospital in which the subject information is obtained, and a transmission unit which transmits the subject information stored in the storage unit and positional information indicating the position obtained by the obtaining unit; and a controller that performs control for storing the subject information in association with identification information for identifying the subject for each subject in a storage device based on the positional information transmitted from the transmission unit of the wearable device.

In the hospital support system of the first aspect, in a hospital support system a second aspect of the present disclosure, the controller performs control for storing the subject information and the identification information further in association with the positional information in the storage device.

In the hospital support system of the first or second aspect, in a hospital support system a third aspect of the present disclosure, the storage unit stores the subject information in a format of image information.

In the hospital support system of the third aspect, in a hospital support system a fourth aspect of the present disclosure, the format of the image information is Portable Document Format (PDF).

In the hospital support system of any one of the first to fourth aspects, in a hospital support system a fifth aspect of the present disclosure, the storage device stores different kinds of information items on the subject for each subject, and the controller classifies the kind of the subject information based on the positional information.

In the hospital support system of any one of the first to fifth aspects, a hospital support system a sixth aspect of the present disclosure further comprises: a display device that displays information on the subject. The controller performs control for storing the identification information corresponding to the information on the subject displayed on the display device in association with the subject information transmitted from the wearable device in the storage device.

In the hospital support system of the sixth aspect, in a hospital support system a seventh aspect of the present disclosure, the information on the subject displayed on the display device includes the identification information.

In the hospital support system of the sixth or seventh aspect, in a hospital support system an eighth aspect of the present disclosure, in a case where a distance between the wearable device and the display device is equal to or shorter than a predetermined distance, the controller performs control for storing the identification information in association with the subject information transmitted from the transmission unit of the wearable device in the storage device.

In the hospital support system of any one of the first to eighth aspects, a hospital support system a ninth aspect of the present disclosure further comprises: a tag that stores the identification information; and a reader that is provided in a predetermined position to read the identification information from the tag. In a case where the reader reads the identification information from the tag, the controller performs control for storing the identification information stored in the tag in association with the subject information transmitted from the transmission unit of the wearable device in the storage device.

In the hospital support system of any one of the first to ninth aspects, in a hospital support system a tenth aspect of the present disclosure, the wearable device is an electronic pen, and the storage unit stores information described for the subject, as the subject information.

In the hospital support system of any one of the first to ninth aspects, in a hospital support system an eleventh aspect of the present disclosure, the wearable device is a camera capable of being attached to eyeglasses or a camera integrated with eyeglasses, and the storage unit stores an image captured for the subject, as the subject information.

A hospital support system of a twelfth aspect of the present disclosure comprises: a wearable device that includes a storage unit which stores obtained subject information on a subject and a transmission unit which transmits the subject information stored in the storage unit; and a reception device that includes a reception unit which receives the subject information transmitted from the transmission unit of the wearable device and a controller which performs control for storing the subject information received by the reception unit in association with identification information for identifying the subject for each subject in a storage device based on positional information indicating a position in which the reception unit receives the subject information.

A hospital support method of a thirteenth aspect of the present disclosure comprises: causing a wearable device to store obtained subject information on a subject, obtain a position within a hospital in which the subject information is obtained, and transmit the stored subject information and positional information indicating the obtained position; and causing a controller to perform control for storing the subject information in association with identification information for identifying the subject for each subject in a storage device based on the positional information transmitted from the wearable device.

A control device of a fourteenth aspect of the present disclosure comprises: a reception unit that receives positional information transmitted from a transmission unit of a wearable device that includes a storage unit which stores obtained subject information on a subject, an obtaining unit which obtains a position within a hospital in which the subject information is obtained, and the transmission unit which transmits the subject information stored in the storage unit and the positional information indicating the position obtained by the obtaining unit; and a controller that performs control for storing the subject information in association with identification information for identifying the subject for each subject in a storage device based on the positional information received by the reception unit.

In order to achieve the object, a non-transitory computer-readable recording medium which stores a control program of a fifteenth aspect of the present disclosure causes a computer to function as: a reception unit that receives positional information transmitted from a transmission unit of a wearable device that includes a storage unit which stores obtained subject information on a subject, an obtaining unit which obtains a position within a hospital in which the subject information is obtained, and the transmission unit which transmits the subject information stored in the storage unit and the positional information indicating the position obtained by the obtaining unit; and a controller that performs control for storing the subject information in association with identification information for identifying the subject for each subject in a storage device based on the positional information received by the reception unit.

According to the present disclosure, it is possible to easily recognize the position in which the information on the subject is obtained by using the wearable device.

### BRIEF DESCRIPTION OF THE DRAWINGS

Exemplary embodiments according to the technique of the present disclosure will be described in detail based on the following figures, wherein:

FIG. 1 is a plan view showing an example of a configuration of an animal hospital of an embodiment;

FIG. 2 is a block diagram showing an example of a configuration of a hospital support system of the embodiment;

FIG. 3 is a block diagram showing an example of a hardware configuration of an operation terminal of the embodiment;

FIG. 4 is a block diagram showing an example of a hardware configuration of an electronic pen of the embodiment;

FIG. 5 is a block diagram showing an example of functional configurations of the operation terminal and the electronic pen of the embodiment;

FIG. 6 is a block diagram showing an example of a hardware configuration of an examination management terminal of the embodiment;

FIG. 7 is a block diagram showing an example of hardware configurations of a waiting room monitor, a check-up monitor, and a management monitor of the embodiment;

FIG. 8 is a flowchart showing an example of control processing performed by the operation terminal of the embodiment;

FIG. 9 is a plan view showing another example of the configuration of the animal hospital of the embodiment;

FIG. 10 is a plan view showing still another example of the configuration of the animal hospital of an embodiment; and

FIG. 11 is a block diagram showing an example of functional configurations of the electronic pen and a reception device of the embodiment shown in FIG. 10.

### DETAILED DESCRIPTION

Hereinafter, an embodiment for implementing a technology of the present disclosure will be described in detail with reference to the drawings.

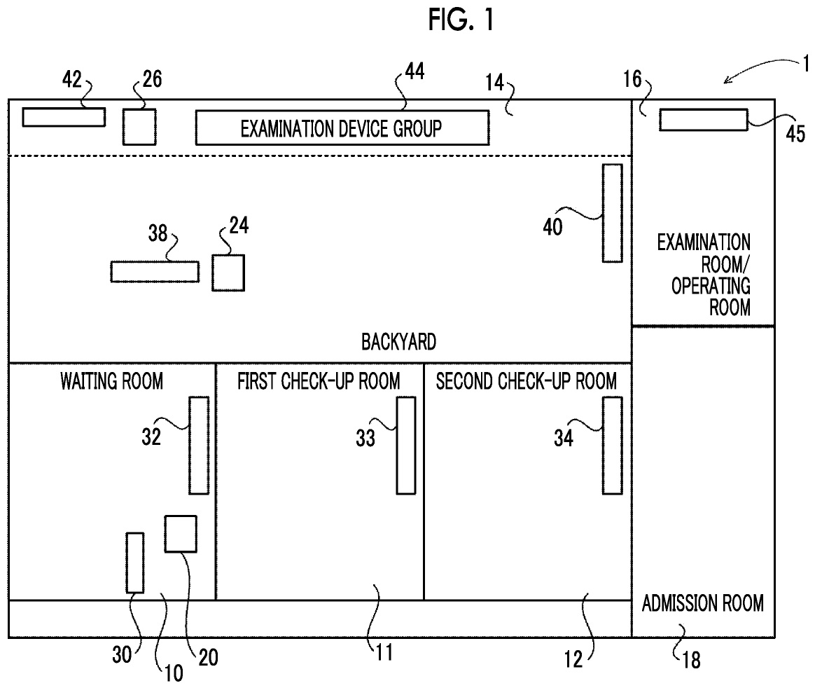

Initially, an example of an animal hospital which is a support target of a hospital support system of the present embodiment will be described with reference to FIG. 1. FIG. 1 shows a plan view of an example of the animal hospital which is the support target of the hospital support system of the present embodiment.

As shown in FIG. 1, an animal hospital 1 is provided with a waiting room 10, a first check-up room 11, a second check-up room 12, a backyard 14, an examination room/operating room 16 that serves as both an examination room and an operating room, and an admission room 18.

In the waiting room 10, a reception for receiving an animal (hereinafter, referred to a “diseased animal” regardless of morbidity) as a subject which visits the animal hospital 1 is provided, and a reception/payment terminal 20 and a reception/payment terminal monitor 30 to be used for receiving the diseased animal or payment by a staff (hereinafter, referred to as a “staff”) such as a veterinarian or a nurse of the animal hospital 1 are disposed. A waiting room monitor 32 for displaying information on contents using an owner of the diseased animal who waits for a check-up as a target to be watched is disposed in the waiting room 10. The waiting room monitor 32 may be a type to display various advertisements related to the diseased animal, information useful for health care of the diseased animal (information on diseases, health diagnosis, pet food, or the like), or an introduction of the animal hospital 1 (an introduction of pet hotels or an introduction related to trimming in a case where the trimming is performed). The waiting room monitor 32 may be a type to display photographs of diseased animals. For example, the waiting room monitor displays one or a plurality of favorite photographs captured by the owner, and thus, it is possible to smoothly facilitate communication between the owners.

A check-up or treatment (curing) on the diseased animal is performed by the veterinarian in each of the first check-up room 11 and the second check-up room 12. A check-up monitor 33 for displaying information on check-up contents or information on the diseased animal such as a so-called medical chart is disposed in the first check-up room 11, and a check-up monitor 34 for the same purpose is disposed in the second check-up room 12. Results of examinations using an examination device group 44 and a radiological examination device 45, for example, a result of a blood examination, a radiographic image, and an ultrasound image are also displayed on the check-up monitor 33 and the check-up monitor 34 of the present embodiment according to a command of the staff.

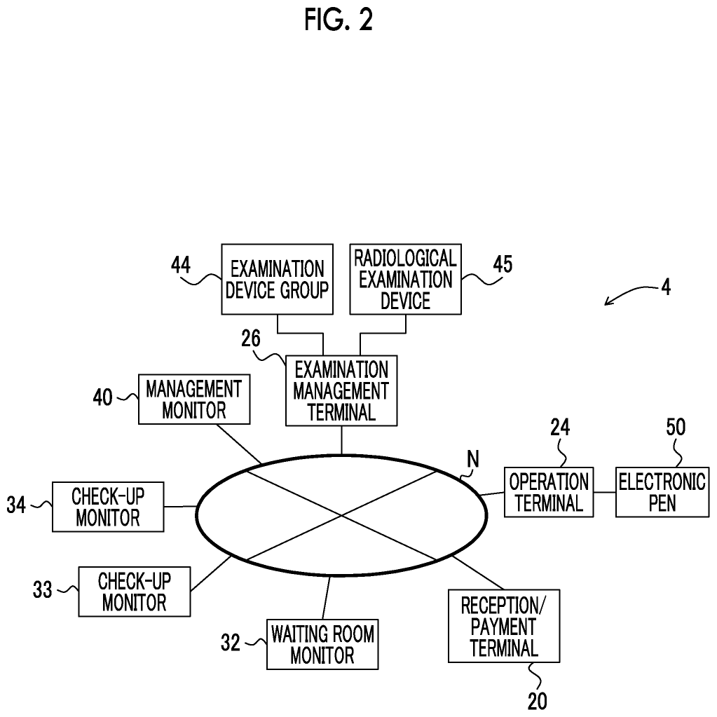

An operation terminal 24, an operation input monitor 38, and a management monitor 40 are provided in the backyard 14, and the entire management of the animal hospital 1 is performed by the staff by using the operation terminal 24, the operation input monitor 38, and the management monitor 40. The operation terminal 24 of the present embodiment controls the entire hospital support system 4 (see FIG. 2) used for managing the animal hospital 1. The operation terminal 24 of the present embodiment is an example of a controller and a control device of the present disclosure. The operation input monitor 38 of the present embodiment is an example of a display device of the present disclosure.

The examination or treatment on the diseased animal or a specimen obtained from the diseased animal is performed by the veterinarian in the backyard 14. Thus, the examination device group 44 used in the examination, an examination management terminal 26 and an examination management monitor 42 for managing the general examination are disposed in the backyard 14. For example, the examination device group 44 includes a blood chemical examination device, an immunological examination device, a radiological examination device, and an ultrasound examination device. The examination device group is not particularly limited, and may include an ultrasound examination device and an endoscopic examination device. As a specific example of each of the blood chemical examination device and the immunological examination device, there is a product name “DRI-CHEM (registered trademark)” manufactured by FUJIFILM Corporation.

In the examination room/operating room 16, the imaging of the radiographic image of the diseased animal or the surgery on the diseased animal is performed by the veterinarian. The radiological examination device 45 for imaging the radiographic image of the diseased animal is disposed in the examination room/operating room 16, and the radiological examination device 45 is managed by using the examination management terminal 26 provided in the backyard 14.

A plurality of diseased animals can be admitted (accommodated) in the admission room 18, and a plurality of animals can stay in the admission room as a so-called pet hotel.

Next, a configuration of the hospital support system 4 of the present embodiment will be described with reference to FIG. 2. As shown in FIG. 2, in the hospital support system 4 of the present embodiment, the reception/payment terminal 20, the operation terminal 24, the examination management terminal 26, the waiting room monitor 32, the check-up monitor 33, the check-up monitor 34, and the management monitor 40 which are stated above are connected to an intra-hospital network N provided within the animal hospital 1 so as to enable communication. The examination management terminal 26, the examination device group 44, and the radiological examination device 45 are connected so as to enable communication without the network N. The communication between the examination management terminal 26, the examination device group 44, and the radiological examination device 45 may be performed in a wireless communication manner or in a wired communication manner. The operation terminal 24 and an electronic pen 50 used for describing subject information on the subject by the staff are connected so as to enable communication without using the network N. The communication between the operation terminal 24 and the electronic pen 50 is performed in a wireless communication manner, and it is particularly preferable that this communication is performed in a short-range wireless communication such as Bluetooth (registered trademark) from the viewpoint of operability and convenience.

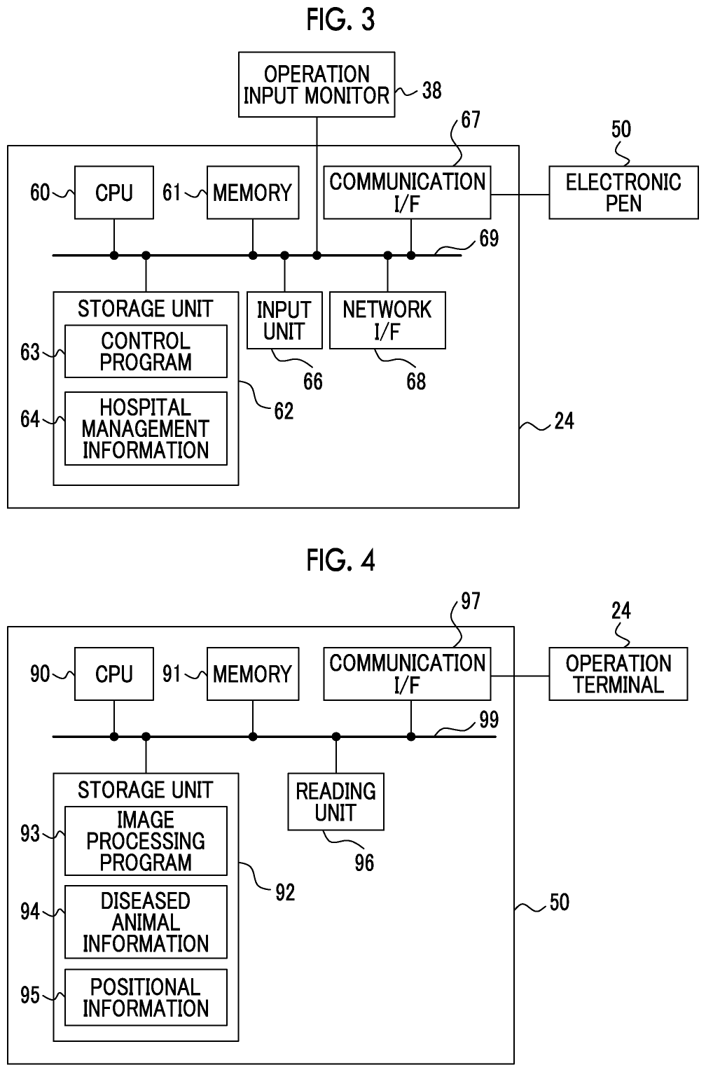

Next, a hardware configuration of the operation terminal 24 of the present embodiment will be described with reference to FIG. 3. As shown in FIG. 3, the operation terminal 24 includes a central processing unit (CPU) 60, a memory 61 as a temporary storage region, and a non-volatile storage unit 62. The operation terminal 24 includes an input unit 66 such as a keyboard or a mouse, a communication I/F 67 to which the electronic pen 50 is connected, and a network interface (I/F) 68 connected to the network N. The CPU 60, the memory 61, the storage unit 62, the input unit 66, the communication I/F 67, the network I/F 68, and the operation input monitor 38 are connected to a bus 69 so as to enable communication.

The storage unit 62 is realized by a hard disk drive (HDD), a solid state drive (SSD), and a flash memory. A control program 63 is stored in the storage unit 62 as a storage medium. The CPU 60 reads out a control program 63 from the storage unit 62, develops the readout program into the memory 61, and executes the developed control program 63. Diseased animal information on the diseased animal which is recorded in a so-called medical chart or hospital management information 64 including various information for managing the entire animal hospital 1 is stored in the storage unit 62. The storage unit 62 of the present embodiment is an example of a storage device of the present disclosure.

A hardware configuration of the electronic pen 50 of the present embodiment will be described with reference to FIG. 4. As shown in FIG. 4, the electronic pen 50 includes a CPU 90, a memory 91 as a temporary storage region, and a non-volatile storage unit 92. The electronic pen 50 includes a communication I/F 97 for communicating with the operation terminal 24, and a reading unit 96 that reads information described in a recording medium such as a sheet by the staff or the owner. The CPU 90, the memory 91, the storage unit 92, the reading unit 96, and the communication I/F 97 are connected to a bus 99 so as to enable communication.

The storage unit 92 is realized by an HDD, an SSD, and a flash memory. An image processing program 93 is stored in the storage unit 92 as the storage medium. The CPU 90 performs image processing by reading out the image processing program 93 from the storage unit 92, developing the readout program into the memory 91, and executing the developed image processing program 93. Diseased animal information 94 and positional information 95 which are described in detail below are stored in the storage unit 92.

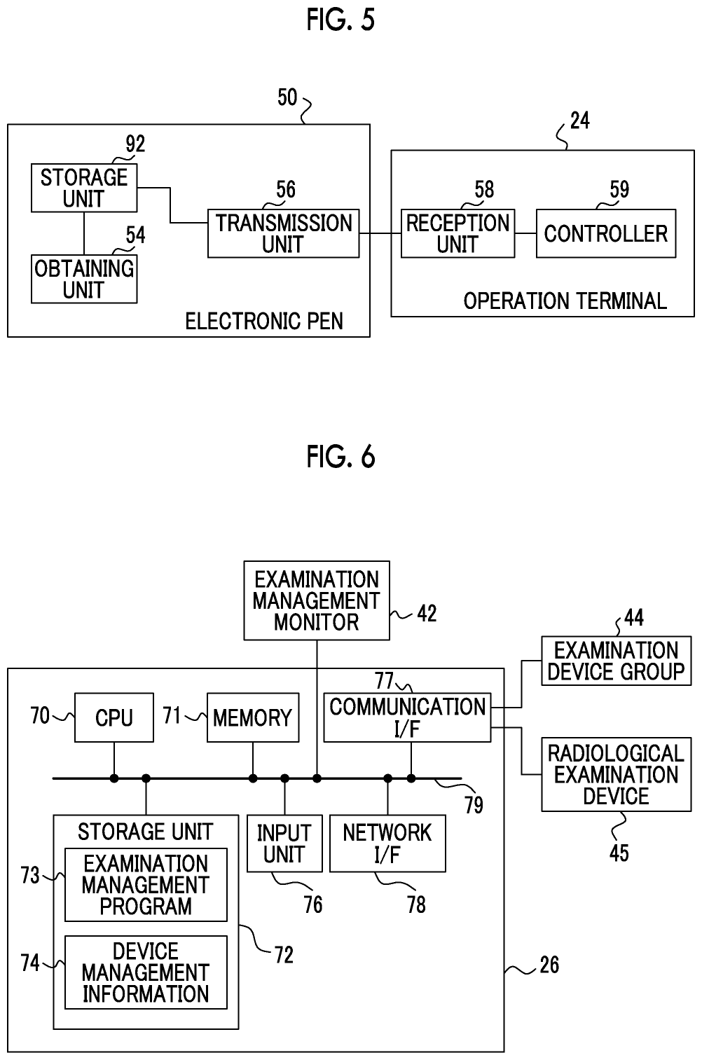

The functional configurations of the operation terminal 24 and the electronic pen 50 of the present embodiment will be described with reference to FIG. 5. As shown in FIG. 5, the electronic pen 50 includes an obtaining unit 54, a transmission unit 56, and a storage unit 92. The CPU 90 functions as the transmission unit 56 by executing the image processing program 93.

The CPU 90 executes the image processing program 93, and thus, the diseased animal information read by the reading unit 96 is stored as the diseased animal information 94 in the storage unit 92 in a format of image information. The method of obtaining the diseased animal information described in the recording medium by using the electronic pen 50 of the present embodiment is not particularly limited. For example, the technology described in JP2004-054375A may be applied to the electronic pen. In the technology described in JP2004-054375A, a dedicated sheet on which a predetermined format (dot pattern) is printed is used as the recording medium, and data corresponding to contents described in the dedicated sheet is obtained by reading the dot pattern. For example, a technology in which the dedicated sheet is not used as the recording medium (the recording medium is not limited) such as a product name of airpenPocket (registered trademark) manufactured by Pentel Co., Ltd. may be applied. In this technology, the data corresponding to the contents described by the electronic pen 50 is obtained by simultaneously transmitting infrared ray and an ultrasound from a pen tip during writing using the electronic pen 50, receiving the infrared ray and the ultrasound reflected by the recording medium, and deriving a position of the pen tip based on a difference in reception time.

In the present embodiment, the CPU 90 executes the image processing as described above. Thus, the format of the image information is obtained by performing the image processing on the information obtained by the obtaining unit 54, and the diseased animal information obtained as the format of the image information is stored as the diseased animal information 94 in the storage unit 92. Examples of the format of the image information include Portable Document Format (PDF), a Joint Photographic Experts Group (JPEG) format, a Graphics Interchange Format (GIF) format, and a Portable Network Graphics (PNG) format, and it is preferable that the PDF is adopted from the viewpoint of ease of handling. The diseased animal information is not limited to the present embodiment, and may be a format on which Optical Character Recognition/Reader (OCR) is performed such as a text format instead of the format of the image information. The image processing in the electronic pen 50 becomes complicated and a processing load becomes large as compared to a case where the format of the image information is adopted.

The obtaining unit 54 of the present embodiment has a function of obtaining a position within the animal hospital 1 in which the diseased animal information 94 is obtained, specifically, the positional information 95 indicating the position. The method of obtaining the positional information 95 by using the obtaining unit 54 is not particularly limited. For example, in a case where the electronic pen 50 comprises a sensor that detects a position such as a Global Positioning System (GPS) sensor, the obtaining unit 54 may obtain the positional information 95 detected by the sensor. For example, the information indicating the position may be described by the staff by using the electronic pen 50, and the positional information 95 may be obtained according to the information indicating the position read by the reading unit 96. The positional information 95 obtained by the obtaining unit 54 is stored in the storage unit 92 in association with the diseased animal information 94.

The transmission unit 56 has a function of transmitting the diseased animal information 94 and the positional information 95 stored in the storage unit 92 to the operation terminal 24 through the communication I/F 97.

Meanwhile, the operation terminal 24 includes a reception unit 58 and a controller 59, as shown in FIG. 5. The reception unit 58 has a function of receiving the diseased animal information 94 and the positional information 95 transmitted from the transmission unit 56 of the electronic pen 50 through the communication I/F 67. The controller 59 has a function of storing, as the hospital management information 64, the diseased animal information 94 received by the reception unit 58 in association with identification information of the diseased animal corresponding to the diseased animal information 94 in the storage unit 62 for each diseased animal based on the positional information 95.

A hardware configuration of the examination management terminal 26 of the present embodiment will be described with reference to FIG. 6. As shown in FIG. 6, the examination management terminal 26 includes a CPU 70, a memory 71 as a temporary storage region, and a non-volatile storage unit 72. The examination management terminal 26 includes an input unit 76 such as a keyboard or a mouse, a communication I/F 77 for communicating with the examination device group 44 and the radiological examination device 45, and a network I/F 78 connected to the network N. The CPU 70, the memory 71, the storage unit 72, the input unit 76, the communication I/F 77, the network I/F 78, and the examination management monitor 42 are connected to a bus 79 so as to enable communication.

The storage unit 72 is realized by an HDD, an SSD, and a flash memory. An examination management program 73 is stored in the storage unit 72 as the storage medium. The CPU 70 reads out an examination management program 73 from the storage unit 72, develops the readout program into the memory 71, and executes the developed examination management program 73. Device management information 74 for managing the examination device group 44 and the radiological examination device 45 is stored in the storage unit 72.

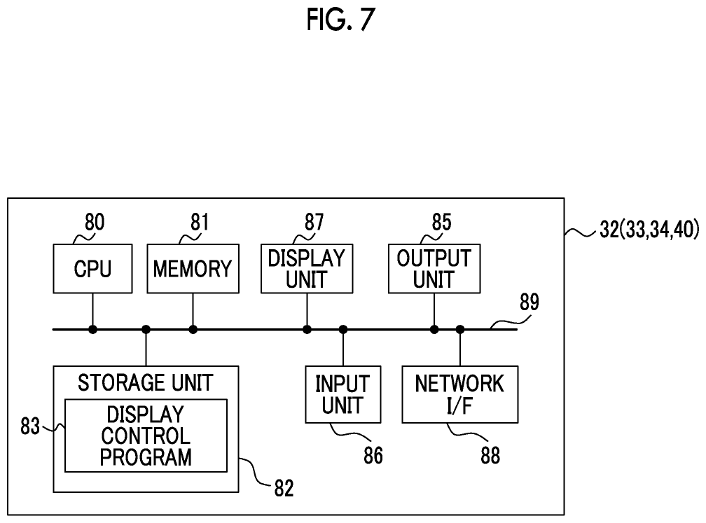

A hardware configuration of the waiting room monitor 32 of the present embodiment will be described with reference to FIG. 7. As shown in FIG. 7, the waiting room monitor 32 includes a CPU 80, a memory 81 as a temporary storage region, and a non-volatile storage unit 82. The waiting room monitor 32 includes an output unit 85 such as a speaker that outputs voice, an input unit 86 such as a remote controller, a display unit 87 such as liquid crystal display, and a network I/F 88 connected to the network N. The CPU 80, the memory 81, the storage unit 82, the output unit 85, the input unit 86, the display unit 87, and the network I/F 88 are connected to a bus 89 so as to enable communication.

The storage unit 82 is realized by an HDD, an SSD, and a flash memory. A display control program 83 is stored in the storage unit 82 as the storage medium. The CPU 80 reads out the display control program 83 from the storage unit 82, develops the readout program into the memory 81, and executes the developed display control program 83. The CPU 80 performs control for displaying various information on the display unit 87 according to the control of the operation terminal 24 by executing the display control program 83.

The hardware configurations of the check-up monitor 33, the check-up monitor 34, and the management monitor 40 are the same as that of the waiting room monitor 32, and thus, the description thereof will be omitted.

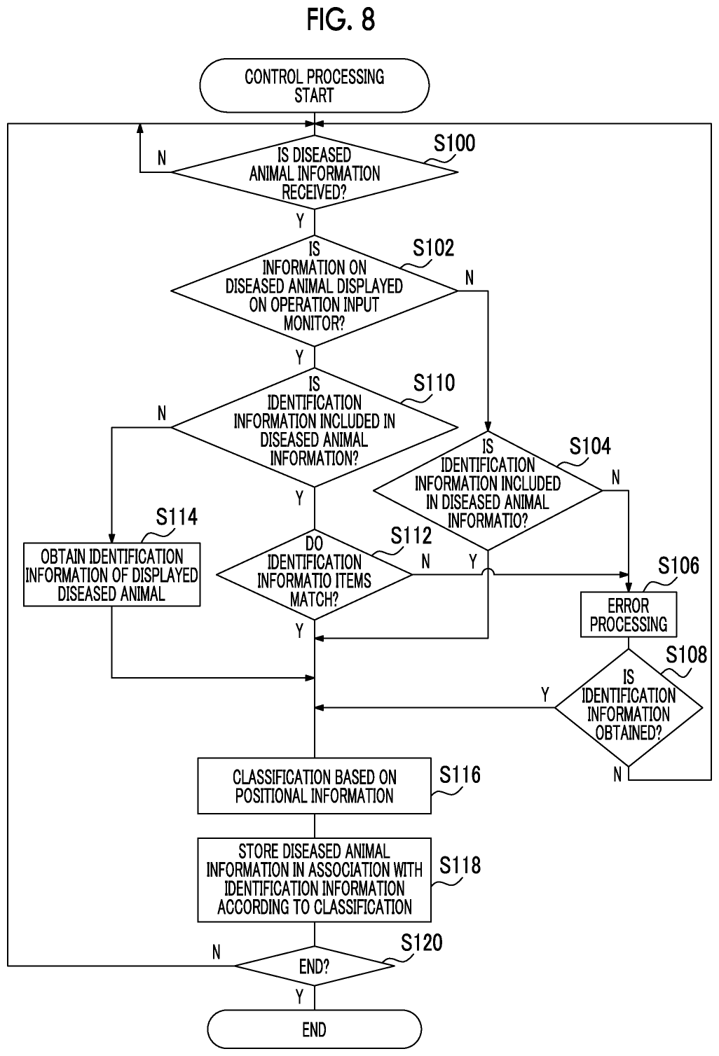

Next, the actions and effects of the hospital support system 4 of the present embodiment will be described. In the present embodiment, the CPU 60 of the operation terminal 24 executes the control program 63, and thus, control processing shown in FIG. 8 is performed. The CPU 60 performs the control processing, and thus, the diseased animal information 94 described by the staff by using the electronic pen 50 is stored in the storage unit 62 of the operation terminal 24. For example, the control processing shown in FIG. 8 is performed at a timing when a power supply (not shown) is input to the operation terminal 24.

In step S100, the CPU 60 determines whether or not the reception unit 58 receives the diseased animal information 94 transmitted from the transmission unit 56 of the electronic pen 50. The determination in step S100 is a negative determination until the diseased animal information 94 is received. In the present embodiment, for example, in a case where a distance between the operation terminal 24 (operation input monitor 38) and the electronic pen 50 is equal to or shorter than a predetermined distance, the diseased animal information 94 and the positional information 95 associated with the diseased animal information 94 are transmitted to the operation terminal 24 from the transmission unit 56 of the electronic pen 50. In a case where the reception unit 58 of the operation terminal 24 receives the diseased animal information 94, the determination in step S100 is a positive determination, and the CPU proceeds to step S102.

In step S102, the CPU 60 determines whether or not the information on the diseased animal is displayed on the operation input monitor 38. In a case where the information on the diseased animal is not displayed on the operation input monitor 38, the determination in step S102 is a negative determination, and the CPU proceeds to step S104.

In step S104, the CPU 60 determines whether or not the identification information is included in the received diseased animal information 94. For example, in a case where the identification information is included in the diseased animal information 94 such as a case where the identification information as the diseased animal information 94 is described by the staff by the electronic pen 50, the determination in step S104 is a positive determination, and the CPU proceeds to step S116. Meanwhile, in a case where the identification information is not included in the diseased animal information 94, the determination in step S104 is a negative determination, and the CPU proceeds to step S106.

In step S106, the CPU 60 performs predetermined error processing. In the hospital support system 4 of the present embodiment, since the diseased animal information 94 in association with the identification information is stored as the hospital management information 64 in the storage unit 62 of the operation terminal 24, in a case where the identification information is not able to be obtained, an error occurs. Thus, the CPU 60 performs the predetermined error processing, but may perform processing for enabling the obtaining of the identification information. Detailed processing contents are not particularly limited, and processing for displaying information indicating that the identification information is not able to be obtained (is not known) on the operation input monitor 38 may be performed. In this case, it is preferable that the staff displays a command for inputting the identification information by using the input unit 66 of the operation terminal 24 on the operation input monitor 38.

Through the error processing in the present step, the CPU 60 obtains the input identification information in a case where the identification information is input by the staff.

In the next step S108, the CPU 60 determines whether or not the identification information is obtained as a result of the error processing in step S106. In a case where identification information is obtained, the determination in step S108 is a positive determination, and the CPU proceeds to step S116. Meanwhile, in a case where the identification information is not obtained even though the error processing is performed, the CPU returns to step S100 without storing the received diseased animal information 94 in the storage unit 62. In the present embodiment, the diseased animal information 94 is stored in the storage unit 92 of the electronic pen 50 with no change in this case.

Meanwhile, in a case where the identification information is included in the diseased animal information 94, the determination in step S104 is a positive determination, and the CPU proceeds to step S116.

Meanwhile, in a case where the information on the diseased animal is displayed on the operation input monitor 38, the determination in step S102 is a positive determination, and the CPU proceeds to step S110. In step S110, the CPU 60 determines whether or not the identification information is included in the received diseased animal information 94, similarly to step S104.

In a case where the identification information is included in the diseased animal information 94, the determination in step S110 is a positive determination, and the CPU proceeds to step S112. In step S112, the CPU 60 determines the identification information of the diseased animal corresponding to the information displayed on the operation input monitor 38 matches the identification information included in the diseased animal information 94. The method of obtaining the identification information of the diseased animal corresponding to the information displayed on the operation input monitor 38 by using the CPU 60 is not particularly limited. In a case where the identification information is displayed on the operation input monitor 38, the identification information may be obtained based on the displayed information. In a case where the identification information is not displayed on the operation input monitor 38, the identification information corresponding to the diseased animal of which the information is displayed may be obtained while referring to the hospital management information 64. In a case where both the identification information items match each other, the determination in step S112 is a positive determination, and the CPU proceeds to step S116. Meanwhile, in a case where both the identification information items do not match each other (in a case where both the identification information items mismatch each other), the determination in step S112 is a negative determination, and the CPU proceeds to step S106. As stated above, in step S106, processing for enabling the obtaining of appropriate identification information is performed by performing the predetermined error processing.

Meanwhile, in a case where the identification information is not included in the diseased animal information 94, the determination in step S110 is a negative determination, the CPU proceeds to step S114. In step S114, the CPU 60 obtains the identification information of the diseased animal corresponding to the information displayed on the operation input monitor 38, and proceeds to step S116.

In step S116, the CPU 60 classifies the kind of the diseased animal information 94 based on the received positional information 95. Multiple kinds of diseased animal information items 94 are stored as the hospital management information 64 of the storage unit 62 of the present embodiment, and any of the multiple kinds of diseased animal information items is associated with the position in which the obtaining unit 54 obtains the diseased animal information 94.

Specifically, for example, in a case where the owner describes an interview sheet by using the electronic pen 50 in the waiting room 10, information indicating contents of the interview sheet in association with the positional information 95 indicating the waiting room 10 is stored in the storage unit 92 of the electronic pen 50, as the diseased animal information 94. Thus, in this case, in a case where the positional information 95 indicates the waiting room 10, the kind of the diseased animal information 94 can be classified as “interview sheet”.

For example, in a case where the veterinarian describes the medical chart by using the electronic pen 50 in the first check-up room 11, information indicating contents of the medical chart in association with the positional information 95 indicating the first check-up room 11 is stored in the storage unit 92 of the electronic pen 50, as the diseased animal information 94. In a case where the veterinarian describes the medical chart by using the electronic pen 50 in the second check-up room 12, information indicating contents of the medical chart in association with the positional information 95 indicating the second check-up room 12 is stored in the storage unit 92 of the electronic pen 50, as the diseased animal information 94. Thus, in this case, in a case where the positional information 95 indicates the first check-up room 11 or the second check-up room 12, the kind of the diseased animal information 94 can be classified as “medical chart”.

For example, in a case where the staff describes examination information on the examination such as the examination result by using the electronic pen 50 in the backyard 14, the examination information in association with the positional information 95 indicating the backyard 14 is stored in the storage unit 92 of the electronic pen 50, as the diseased animal information 94. Thus, in this case, in a case where the positional information 95 indicates the backyard 14, the kind of the diseased animal information 94 can be classified as “examination information”.

In the next step S118, the CPU 60 stores the diseased animal information 94 in association with the identification information in the storage unit 62 of the operation terminal 24 according to the classification in step S116, as the hospital management information 64. The CPU 60 may describe the diseased animal information 94 in association with the positional information 95 in the storage unit 62 in storing the diseased animal information in the storage unit 62.

In the next step S120, the CPU 60 determines whether or not to end the present control processing. For example, until a switch (not shown) of the operation terminal 24 is turned off, the determination in step S120 is a negative determination, and the CPU returns to step S100 and repeats the processing in step S100 to S118. Meanwhile, for example, in a case where the switch of the operation terminal 24 is turned off, the determination in step S120 is a positive determination, and the present control processing is ended.

As described above, the hospital support system 4 of the present embodiment includes the electronic pen 50 that includes the storage unit 92 which stores the obtained diseased animal information 94, the obtaining unit 54 which obtains the position in which the animal hospital 1 in which the diseased animal information 94 is obtained, and the transmission unit 56 which transmits the diseased animal information 94 stored in the storage unit 92 and the positional information 95 obtained by the obtaining unit 54. The hospital support system 4 also includes the controller 59 of the operation terminal 24 which performs control for storing, as the hospital management information 64, the diseased animal information 94 in association with the identification information for identifying the subject for each diseased animal in the storage unit 62 of the operation terminal 24 based on the positional information 95 transmitted from the transmission unit 56 of the electronic pen 50.

As stated above, in the hospital support system 4 of the present embodiment, the diseased animal information 94 in association with the identification information is stored in the storage unit 62 of the operation terminal 24 for each diseased animal based on the positional information 95 transmitted from the transmission unit 56 of the electronic pen 50. Therefore, according to the hospital support system 4 of the present embodiment, it is possible to easily recognize the position in which the diseased animal information 94 is obtained by the electronic pen 50.

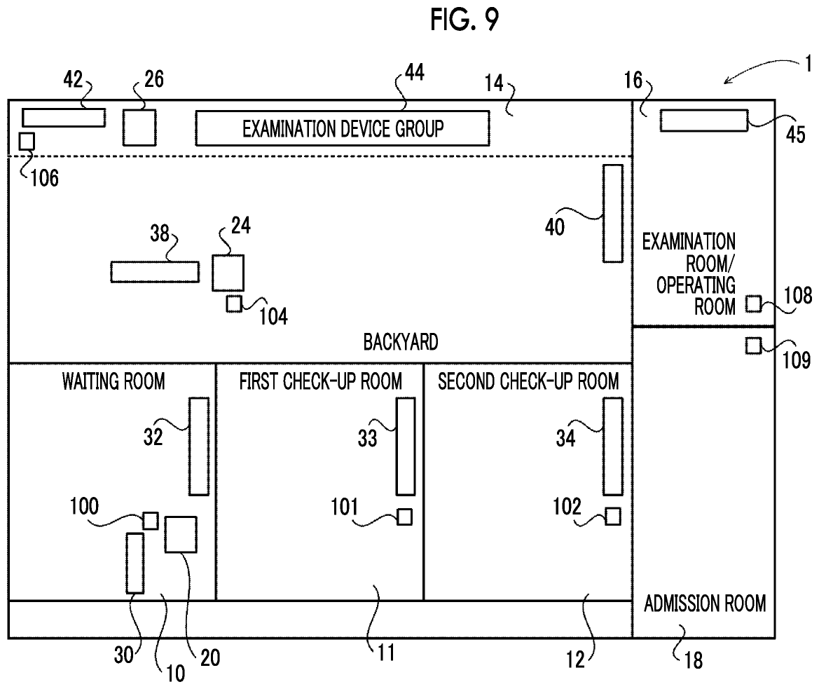

The example in which the electronic pen 50 transmits the diseased animal information 94 and the positional information 95 stored in the storage unit 92 to the operation terminal 24 is not limited to the example described in the present embodiment. For example, an integrated circuit (IC) tag that stores the identification number of the diseased animal and a tag reader that functions as a reader for reading the identification information from the IC tag may be used. For example, in this example, a tag reader 100, a tag reader 101, a tag reader 102, tag readers 104 and 106, a tag reader 108, and a tag reader 109 in association with positions (rooms) are respectively disposed in the waiting room 10, the first check-up room 11, the second check-up room 12, the backyard 14, the examination room/operating room 16, and the admission room 18, as in an example shown in FIG. 9. The electronic pen 50 in a state in which the diseased animal information 94 is stored in the storage unit 92 and the integrated circuit (IC) tag that stores the identification number of the diseased animal are integrated by using a binder, and thus, the identification information of the IC tag is read by any of the tag readers 100, 101, 102, 104, 106, 108, and 109. In a case where the identification information is read by any of the tag readers 100, 101, 102, 104, 106, 108, and 109, the read identification information, the positional information 95 associated with the tag reader that reads the identification information, the diseased animal information 94 stored in the storage unit 92 of the electronic pen 50 in association with each other may be transmitted to the operation terminal 24.

The IC tag may be included in a check-up card of the diseased animal, or a so-called IC tag seal capable of being pasted on the medical chart may be used as the IC tag. A near field communication (NFC) tag, a seal on which two-dimensional barcode is printed, or a GPS tag may be adopted instead of the IC tag or in addition to the IC tag. In a case where the GPS tag is adopted and it is detected that the GPS tag approaches the operation terminal 24 based on the positional information 95 obtained from the GPS tag, since the reader such as the tag reader may not be provided, it is preferable that the identification information, the positional information 95, and the diseased animal information 94 stored in the storage unit 92 of the electronic pen 50 in association with each other are transmitted to the operation terminal 24.

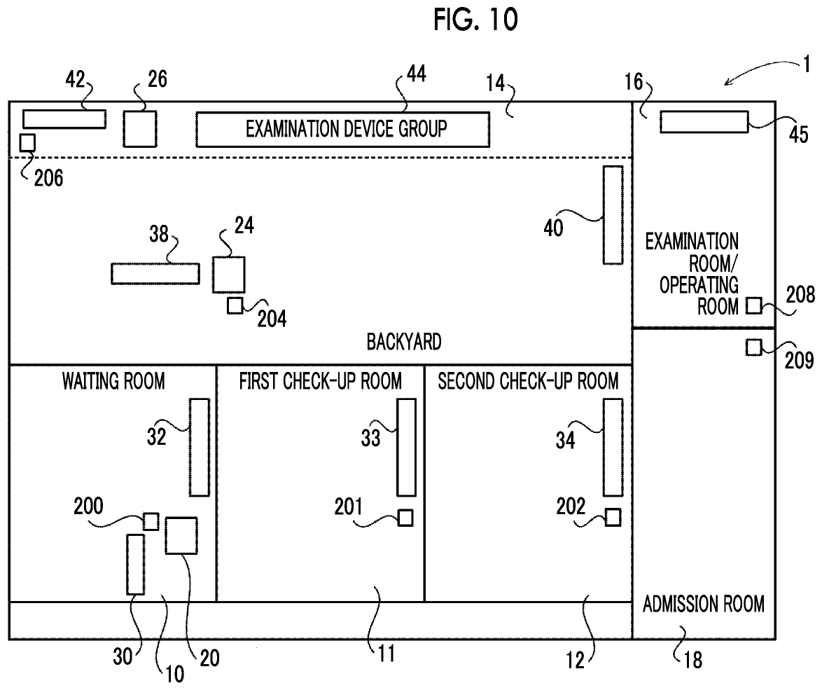

Although it has been described in the present embodiment that the positional information 95 obtained the obtaining unit 54 of the electronic pen 50 is used as the positional information 95, the method of obtaining the positional information 95 is not limited to the present embodiment. For example, the diseased animal information 94 may be transmitted but the positional information 95 may not be transmitted from the transmission unit 56 of the electronic pen 50. A reception device that receives the diseased animal information 94 transmitted from the transmission unit 56 of the electronic pen 50 and stores the received information in the storage unit 62 of the operation terminal 24 may be used. For example, in the case of the hospital support system 4 in this example, a reception device 200, a reception device 201, reception device 202, reception devices 204 and 206, a reception device 208, and a reception device 209 in association with positions (rooms) are respectively disposed in the waiting room 10, the first check-up room 11, the second check-up room 12, the backyard 14, the examination room/operating room 16, and the admission room 18, as in an example shown in FIG. 10.

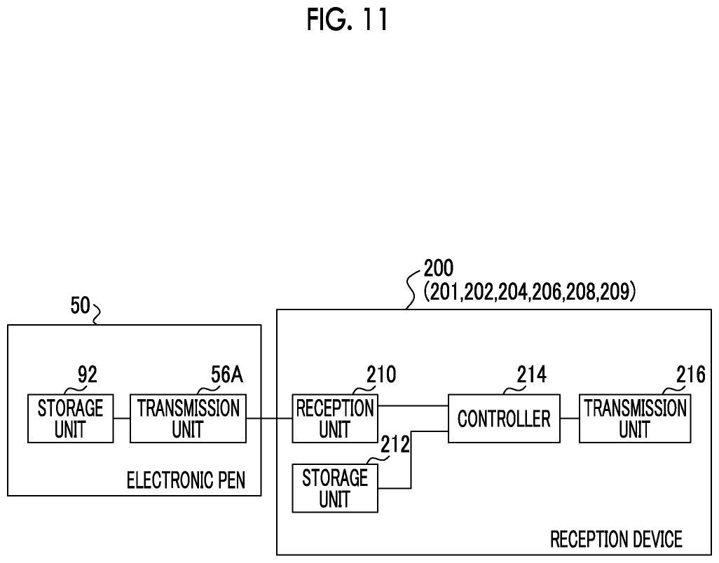

Functional configurations of the electronic pen 50 and the reception devices 200, 201, 202, 204, 206, 208, and 209 in this case will be described with reference to FIG. 11.

As shown in FIG. 11, the electronic pen 50 includes a transmission unit 56A, and the storage unit 92 that stores the diseased animal information 94. The transmission unit 56 has a function of transmitting the diseased animal information 94 stored in the storage unit 92 to the operation terminal 24 through the communication I/F 97.

Meanwhile, as shown in FIG. 11, the reception device 200 includes a reception unit 210, a storage unit 212, a controller 214, and a transmission unit 216. The reception unit 210 has a function of receiving the diseased animal information 94 transmitted from the transmission unit 56A of the electronic pen 50 through a communication I/F (not shown). The storage unit 212 has a function of storing the positional information 95 indicating the position of the reception device. A sensor that detects a position such as a GPS sensor which detects the position of the reception device may be provided instead of the storage unit 212.

The controller 214 has a function of performing control for obtaining the positional information 95 indicating the position in which the reception unit 210 receives the diseased animal information 94 transmitted from the transmission unit 56A of the electronic pen 50 from the storage unit 212, transmitting the diseased animal information 94 in association with the positional information 95 to the operation terminal 24 from the transmission unit 216, and storing the diseased animal information 94 in association with the identification information for each diseased animal in the storage unit 62 of the operation terminal 24 based on the positional information 95.

Since the functional configuration of each of the reception device 201, 202, 204, 206, 208, and 209 is the same as the functional configuration of the reception device 200, the description thereof will be omitted.

In the hospital support system 4 in this case, the diseased animal information 94 in association with the identification information is stored in the storage unit 62 of the operation terminal 24 for each diseased animal based on the positional information 95 obtained by each of the reception devices 200, 201, 202, 204, 206, 208, and 209 provided in each room within the animal hospital 1 in which the electronic pen 50 is used. Thus, in the hospital support system 4 in this case, it is also possible to easily recognize the position in which the diseased animal information 94 is obtained by using the electronic pen 50.

For example, in a case where the identification information is described in a predetermined position of the recording medium, the controller may transmit the positional information 95 and the diseased animal information 94 stored in the storage unit 92 of the electronic pen 50 in association with the described identification information to the operation terminal 24.

It has been described in the present embodiment that the electronic pen 50 is adopted as an example of a wearable device of the present disclosure. However, the wearable device is not limited to the present embodiment, and may be an electronic device capable of being easily carried by being worn by the owner or the staff. As another example of the wearable device, there is an imaging apparatus such as a camera attachable to eyeglasses worn by the staff or a camera integrated with eyeglasses. In a case where the wearable device includes the camera, the diseased animal information is not limited to the diseased animal information 94 described by the staff. Since the imaging is performed according to a command of the staff, it is possible to record the same scene (state) as a scene (state) viewed by the staff in the format of the image information. A still another example of the wearable device may be a microphone (sound collection device) capable of being worn by the staff. In a case where the microphone is used as the wearable device, the diseased animal information 94 or the identification information may be input by voice instead of the handwriting input using the electronic pen 50. In this case, it is preferable that the diseased animal information 94 input by the voice is recorded as the image information format or the text format by performing processing for recognizing voice (language) information and converting the recognized information into character information from the viewpoint of viewing.

It has been described in the present embodiment that the operation terminal 24 functions as an example of the controller and the control device of the present disclosure. However, the device different from the operation terminal 24, such as the examination management terminal 26, may function as the example of the controller and the control device of the present disclosure instead of the operation terminal 24 or in addition to the operation terminal 24. It has been described in the present embodiment that the storage unit 62 of the operation terminal 24 functions as the example of the storage device of the present disclosure. However, the device different from the storage unit 62, such as a storage device outside the hospital support system 4, may function as the example of the storage device of the present disclosure instead of the storage unit 62 or in addition to the storage unit 62.

Although it has been described in the present embodiment that the animal hospital 1 in which the medical care is performed on the animal other than the person is used as an example of the hospital to which the hospital support system 4 is applied and the animal (diseased animal) other than the person is used as the subject, the present invention is not limited to the present embodiment. The hospital in which the medical care is performed on the person may be used as the example of the hospital to which the hospital support system 4 is applied, and the person may be used as the subject. Similarly to the case of an animal other than a person, since there is a concern that the device will be broken or contaminated due to the rampage of an infant or a patient in the field of emergency medical care, an electronic device such as a tablet terminal or a personal computer may be difficult to be used in the medical care. Thus, even in a case where the hospital in which the medical care is performed on the person is the hospital to which the hospital support system 4 is applied, it is possible to obtain an effect of easily recognizing the position in which the information on the diseased animal information 94 is obtained by using the wearable device, as stated above.

Various processing performed by the CPU executing the software (program) in the aforementioned embodiment may be performed by various processors other the CPU. As the processor in this case, there are a programmable logic device (PLD) capable of changing a circuit configuration after Field-Programmable Gate Array (FPGA) is manufactured and a dedicated electric circuit which is a processor having a circuit configuration designed as a dedicated circuit in order to perform specific processing such as an Application Specific Integrated Circuit (ASIC). The various processing may be performed by one of the various processors, or may be performed by a combination of the same kind or different kinds of two or more processors (for example, a plurality of FPGAs, and a combination of the CPU and the FPGA). More specifically, the hardware configurations of the various processors are electric circuits in which circuit elements such as semiconductor elements are combined.

Although it has been described in the aforementioned embodiment that the control program 63 is stored (installed) in the storage unit 62 in advance, the image processing program 93 is stored in the storage unit 92 in advance, the examination management program 73 is stored in the storage unit 72 in advance, and the display control program 83 is stored in the storage unit 82 in advance, the present invention is not limited thereto. Each of the control program 63, the image processing program 93, the examination management program 73, and the display control program 83 may be stored in a recording medium such as a compact disk read only memory (CD-ROM), a digital versatile disk read only memory (DVD-ROM), and universal serial bus (USB) memory. Each of the control program 63, the image processing program 93, the examination management program 73, and the display control program 83 may be downloaded from an external device through a network.

What is claimed is:

1. A hospital support system comprising:
a wearable device that includes a storage unit which stores obtained subject information on a subject, an obtaining unit which obtains a position within a hospital in which the subject information is obtained, and a transmission unit which transmits the subject information stored in the storage unit and positional information indicating the position obtained by the obtaining unit; and
a controller that performs control for storing the subject information in association with identification information for identifying the subject for each subject in a storage device based on the positional information transmitted from the transmission unit of the wearable device.

2. The hospital support system according to claim 1,
wherein the controller performs control for storing the subject information and the identification information further in association with the positional information in the storage device.

3. The hospital support system according to claim 1,
wherein the storage unit stores the subject information in a format of image information.

4. The hospital support system according to claim 3,
wherein the format of the image information is Portable Document Format (PDF).

5. The hospital support system according to claim 1,
wherein the storage device stores different kinds of information items on the subject for each subject, and the controller classifies the kind of the subject information based on the positional information.

6. The hospital support system according to claim 1, further comprising:
a display device that displays information on the subject,
wherein the controller performs control for storing the identification information corresponding to the information on the subject displayed on the display device in association with the subject information transmitted from the wearable device in the storage device.

7. The hospital support system according to claim 6,
wherein the information on the subject displayed on the display device includes the identification information.

8. The hospital support system according to claim 6,
wherein, in a case where a distance between the wearable device and the display device is equal to or shorter than a predetermined distance, the controller performs control for storing the identification information in association with the subject information transmitted from the transmission unit of the wearable device in the storage device.

9. The hospital support system according to claim 1, further comprising:
a tag that stores the identification information; and
a reader that is provided in a predetermined position to read the identification information from the tag,
wherein, in a case where the reader reads the identification information from the tag, the controller performs control for storing the identification information stored in the tag in association with the subject information transmitted from the transmission unit of the wearable device in the storage device.

10. The hospital support system according to claim 1,
wherein the wearable device is an electronic pen, and the storage unit stores information described for the subject, as the subject information.

11. The hospital support system according to claim 1,
wherein the wearable device is a camera capable of being attached to eyeglasses or a camera integrated with eyeglasses, and the storage unit stores an image captured for the subject, as the subject information.

12. A hospital support system comprising:
a wearable device that includes a storage unit which stores obtained subject information on a subject and a transmission unit which transmits the subject information stored in the storage unit; and
a reception device that includes a reception unit which receives the subject information transmitted from the transmission unit of the wearable device and a controller which performs control for storing the subject information received by the reception unit in association with identification information for identifying the subject for each subject in a storage device based on positional information indicating a position in which the reception unit receives the subject information.

13. A hospital support method comprising:
causing a wearable device to store obtained subject information on a subject, obtain a position within a hospital in which the subject information is obtained, and transmit the stored subject information and positional information indicating the obtained position; and
causing a controller to perform control for storing the subject information in association with identification information for identifying the subject for each subject in a storage device based on the positional information transmitted from the wearable device.

14. A control device comprising:
a reception unit that receives positional information transmitted from a transmission unit of a wearable device that includes a storage unit which stores obtained subject information on a subject, an obtaining unit which obtains a position within a hospital in which the subject information is obtained, and the transmission unit which transmits the subject information stored in the storage unit and the positional information indicating the position obtained by the obtaining unit; and
a controller that performs control for storing the subject information in association with identification information for identifying the subject for each subject in a storage device based on the positional information received by the reception unit.

15. A non-transitory computer-readable recording medium which stores a control program causing a computer to function as:
a reception unit that receives positional information transmitted from a transmission unit of a wearable device that includes a storage unit which stores obtained subject information on a subject, an obtaining unit which obtains a position within a hospital in which the subject information is obtained, and the transmission unit which transmits the subject information stored in the storage unit and the positional information indicating the position obtained by the obtaining unit; and
a controller that performs control for storing the subject information in association with identification information for identifying the subject for each subject in a storage device based on the positional information received by the reception unit.
